(19) United States
(12) Patent Application Publication
(10) Pub. No.: US 2026/0174896 A1
(43) Pub. Date: Jun. 25, 2026

(54) VESICLES COMPRISING SECRETORY PROTEINS EXPRESSED ON THE SURFACE AND USES THEREOF FOR DELIVERY OF AN AGENT TO CELLS UNDER CELLULAR STRESS AND REGULATED CELL DEATH

(71) Applicant: TZU CHI UNIVERSITY, Hualien City (TW)

(72) Inventors: Hsin-Hou Chang, Hualien City (TW); Der-Shan Sun, Hualien City (TW)

(21) Appl. No.: 18/990,271

(22) Filed: Dec. 20, 2024

(51) Int. Cl. A61K 47/69; A61K 45/06; A61K 47/68

(52) U.S. Cl. CPC A61K 47/6913; A61K 45/06; A61K 47/6849

(57) ABSTRACT

The present disclosure provides a protein-vesicle conjugate, comprising one or more secretory proteins or a fragment thereof expressed on or conjugated to a surface of a vesicle and optionally an agent, and a method for delivery of an agent with the protein-vesicle conjugate to an autophagic and/or apoptotic cell and a tissue containing the cell.

### FIELD OF THE INVENTION

The present invention relates to agent delivery to cells, and particularly to delivery of agents to a cell under cellular stress and regulated cell death.

### BACKGROUND OF THE INVENTION

Cellular stress and regulated cell death (RCD) are vital for maintaining cellular homeostasis and adapting to environmental changes. Various factors, such as toxic agents, physical impacts, oxidative stress, DNA damage, nutrient deprivation, and metabolic dysregulations, can induce cellular stress. In response, cells activate signaling pathways that can lead to adaptive changes or, if the damage is irreparable, to RCD. RCD mechanisms, including apoptosis, autophagy, necroptosis, pyroptosis, and ferroptosis, help eliminate damaged or infected cells, preventing them from becoming cancerous or contributing to inflammatory diseases, and ultimately leading to tissue repair and homeostasis under physiological conditions.

For instance, apoptosis, a well-known form of RCD involving caspase activation, is crucial for normal development and immune function. Dysregulated apoptosis can result in persistent, potentially immortal cells, as seen in cancer and leukemia, or excessive cell death, causing severe tissue damage in conditions such as strokes and neurodegenerative disorders like Alzheimer's, Huntington's, and Parkinson's diseases. As caspse-3 activation can lead to apoptosis, caspase-3 inhibitor could theoretically be used to rescue apoptosis-associated diseases.

Autophagy, which involves the sequestration of proteins and organelles in double-membrane structures called autophagosomes for lysosomal degradation, is also critical in disease contexts. Beyond cancer and neurodegeneration, modulating autophagy is a therapeutic strategy for various diseases and disorders. For example, certain liver diseases, cardiac diseases, and muscle diseases are associated with the accumulation of misfolded protein aggregates. In these cases, agents that enhance cellular autophagy can promote the clearance of these aggregates, contributing to treatment and reducing disease severity. Conversely, autophagy inhibitors may serve as therapeutic agents in conditions like pancreatitis.

Targeting cells undergoing stress or RCD with a drug delivery system is crucial for therapeutic interventions, as it ensures precise delivery of treatments where they are most needed. Therefore, developing strategies to modulate cellular stress and RCD responses in cells is essential for treating or alleviating stressed-, and RCD-associated diseases.

### SUMMARY OF THE INVENTION

In one aspect, the present disclosure provides a protein-vesicle conjugate, comprising one or more secretory proteins or a fragment thereof expressed on or conjugated to a surface of a vesicle, and/or one or more antibodies specific to the one or more secretory proteins of the fragment thereof and expressed on or conjugated to the surface of the vesicle and optionally an agent.

In one embodiment, the protein-vesicle conjugate comprises one or more secretory proteins or a fragment thereof expressed on or conjugated to a surface of a vesicle and optionally an agent. In another embodiment, the protein-vesicle conjugate comprises one or more antibodies specific to the one or more secretory proteins of the fragment thereof and expressed on or conjugated to the surface of the vesicle and optionally an agent. In another embodiment, the protein-vesicle conjugate comprises one or more secretory proteins or a fragment thereof expressed on or conjugated to a surface of a vesicle, and one or more antibodies specific to the one or more secretory proteins of the fragment thereof and expressed on or conjugated to the surface of the vesicle and optionally an agent.

Examples of the one or more secretory proteins or fragment thereof described herein include, but are not limited to, cation-independent mannose-6-phosphate receptor (CI-MPR), Klotho, programmed death-ligand 1 (PD-L1), intercellular adhesion molecule 1 (ICAM-1; CD54), Toll like receptor 3 (TLR3), IL-1β, IL-1 receptor antagonist (IL-1RA), TNF-α, etanercept (soluble TNF receptor), galectin-8, EGF, FGF23, GDNF, BDNF, β-NGF, NT3, TGF-β1, activin A, inhibitin A, BMP4, BMP6, BMP9, BMP10, GDF-8, GDF-10, GDF-11, GDF-15, and a fragment thereof. In some embodiments, the one or more antibodies specific to the one or more secretory proteins or the fragment thereof may be anti-PSGL-1 and/or anti-PD1 antibodies.

In some embodiments, the protein-vesicle conjugate described herein comprises CI-MPR in combination with Klotho, CI-MPR in combination with P-selectin, or Klotho in combination with P-selectin.

One or more additional lectins or fragment thereof can be used in combination with the protein-vesicle conjugate recited herein. The one or more additional lectins or fragment thereof may also be expressed on or conjugated to the surface of the vesicle. The one or more additional lectins or fragment thereof may include, but are not limited to, P-selectin, E-selectin, L-selectin, P-selectin-ligand-1 (PSGL-1), cation-dependent mannose-6-phosphate receptor (CD-MPR), macrophage mannose receptor (MMR; CD206), galectin 1, galectin 3, annexin V, CD31, integrin αLβ2, VE-cadherin, CD44, CD300a, CD47, CD36, Siglec (CD22), thrombospondin 1 (TSP1), CLEC2, Toll like receptor 4 (TLR4) and a fragment thereof. In further embodiments, the protein-vesicle conjugate comprises CI-MPR or Klotho in combination with P-selectin, E-selectin, PSGL-1, CD31, TLR3, galectin 3, CLEC2, or integrin αLβ2. The protein-vesicle conjugate with the combination of lectins can achieve a synergistic effect on the targeting and drug delivery to autophagic and/or apoptotic cells and autophagic and/or apoptotic cells-containing tissues. The synergistic effect on the targeting effect reduces the effective dosage of the agent to be delivered and thus side effects of the drug can be reduced.

In one embodiment, the agent described herein is separated from the secretory proteins or the fragment thereof and encapsulated within the vesicle or attach to an outer surface of the vesicle.

In some embodiments, the vesicle described herein is a liposome or a micelle.

In some embodiments, the protein-vesicle conjugate described herein can be artificially engineered or cell-derived.

In some embodiments, the agent described herein is a diagnostic contrast agent, a cell survival enhancing agent, a cell survival suppressing agent, a cell component, an organelle, a cell, a cytotoxic agent, an antitumor drug, a toxin or an antibody a lipid, a protein, DNA, RNA, a therapeutic agent or a nanomaterial.

The present disclosure also provides a pharmaceutical composition comprising the protein-vesicle conjugate described herein and a pharmaceutically acceptable carrier.

In some embodiments, the pharmaceutical composition further comprises antagonists to the one or more secretory proteins or a fragment thereof. In some embodiments, the antagonists to the one or more secretory proteins or a fragment thereof are soluble forms of the secretory proteins or fragments thereof, corresponding ligands to the secretory proteins or fragments thereof, or antibodies to the secretory proteins or fragments thereof.

In another aspect, the present disclosure provides a method for targeting delivery of an interested agent to an autophagic and/or apoptotic cell or a tissue containing the cell, comprising administering the protein-vesicle conjugate to a subject. In one embodiment, before administration of the vesicle, the method additionally comprises a step of administering an autophagic and/or apoptotic inducing agent to a target cell or tissue.

In one embodiment, before administration of the protein-vesicle conjugate, the method further comprises a step of administering an autophagic and/or apoptotic-inducing agent to a target cell or a target tissue.

In one embodiment, the method further comprises a step of administering an autophagic and/or apoptotic-inducing agent to a target cell or a target tissue.

In one embodiment, the the delivery of the agent with the protein-vesicle conjugate described herein to the autophagic cell is directed to a disease associated with autophagy deregulation and/or apoptosis alteration.

In some embodiments, the disease associated with autophagy deregulation is trauma, exposure to chemical and physical toxic factors, genetic disease, age-related disease, cardiovascular disease, infectious disease, neoplastic disease, neurodegenerative disease, metabolic disease, aging, obesity, cancer, neurodegeneration induced by β-amyloid or a-synuclein or toxicity, myodegenerative conditions, or chronic lung inflammation caused by cystic fibrosis.

In some embodiments, the disease associated with apoptosis alteration is trauma, exposure to chemical and physical toxic factors, genetic disease, age-related disease, age-related disease, cardiovascular disease, infectious disease, neoplastic disease, neurodegenerative disease, metabolic disease, aging, obesity, cancer, neurodegeneration induced by β-amyloid or a-synuclein or toxicity, myodegenerative conditions, or chronic lung inflammation caused by cystic fibrosis, or autoimmune disease.

### BRIEF DESCRIPTION OF DRAWINGS

The patent or application file contains at least one drawing executed in color. Copies of this patent or patent application publication with color drawings(s) will be provided by the Office upon request and payment of the necessary fee.

FIG. 1 illustrates the targeting of inflamed and dying cells in TAA-treated mice using fluorescein-loaded liposomes conjugated with secretory proteins. FIG. 1A outlines the experimental design. FIGS. 1B and 1C present lists of secretory proteins and proteins used for liposome conjugation (upper panels) and the representative fluorescence images of the mouse liver (lower panels).

FIG. 2 presents the quantitative results of fluorescence intensities for various lectin-conjugated fluorescein-loaded liposomes targeting inflamed and dying cells in TAA-treated mice. FIG. 2A provides an overview of the experiment. FIG. 2B shows quantitative analysis of mouse liver images after the intravenous administration of the fluorescein-loaded liposomes. Groups with fluorescence intensity above the solid line (equivalent to the control groups treated with BSA and IgG-Fc conjugated liposomes) were considered to provide effective targeted delivery. The fluorescence intensity of the vehicle group was normalized to 1-fold.

FIG. 3 illustrate the ameliorative effect of caspase-1 siRNA-loaded liposomes in rescuing inflamed and injured cells in the mouse liver using lectin-conjugated liposomes. FIG. 3A provides an overview of the experiment, while FIG. 3B shows the measurement of relative circulating aspartate transaminase (AST) levels in mouse plasma after TAA treatment. The results are compared to controls, quantifying the relative circulating AST levels following the intravenous administration of liposomes conjugated with various lectin proteins. Groups with circulating AST levels lower than the dashed line (approximately equivalent to the control groups treated with BSA and IgG-Fc conjugated liposomes) were considered to have effective targeted delivery and effective rescue of pyroptosis cell death of the mouse liver (caspase-1 siRNA considered as a pyroptosis inhibitor). The fluorescence intensity of the vehicle group was normalized to 1-fold.

FIG. 4 shows lectin-conjugated (CI-MPR, KL, CI-MPR+KL, CI-MPR+P-selectin- and KL+P-selectin) caspase-3 inhibitor (Z-DEVD-FMK)-loaded liposomes on the rescue of TAA-induced hepatitis in mice. The plasma aspartate transaminase (AST) levels were analyzed. P-selectin: P-sel; Klotho protein: KL; cation-independent mannose-6-phosphate receptor: CI-MPR, #P<0.05, ##P<0.01, ###P<0.001, compared to untreated control groups. +P<0.05, ++P<0.01, compared to control protein bovine serum albumin (BSA) conjugated groups. * P<0.05, compared to respective single recombinant protein conjugated groups. Groups with circulating AST levels lower than those treated with BSA liposomes were considered to have effective targeted delivery and successful rescue of apoptotic cell death in the mouse liver (with caspase-3 inhibitor serving as an apoptosis inhibitor). Moreover, the combination of multiple lectin conjugations demonstrated more effective amelioration, suggesting synergistic rescue effects.

### DETAILED DESCRIPTION OF THE INVENTION

Where the definition of terms departs from commonly used meaning, the applicant intends to utilize the following definitions, unless specifically indicated.

As used in this specification and the appended claims, the singular forms “a,” “an” and “the” include plural referents unless the content clearly dictates otherwise.

As used herein, the use of “or” means “and/or” unless stated otherwise. In the context of a multiple dependent claim, the use of “or” refers back to more than one preceding independent or dependent claim in the alternative only.

As used herein, the term “one or more” is readily understood by one of skill in the art, particularly when read in context of its usage.

As used herein, the term “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

As used herein the term “micelle” refers to an aggregate (or supramolecular assembly) of surfactant molecules dispersed in a liquid colloid. A typical micelle in aqueous solution forms an aggregate with the hydrophilic “head” regions in contact with surrounding solvent, sequestering the hydrophobic single-tail regions in the micelle center.

As used herein, the terms “agent” or “therapeutic agent” refers to an agent capable of treating and/or ameliorating a condition or disease.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “therapeutically effective amount” or “efficacious amount” refers to the amount of the vesicle that, when administered to a mammal or other subject for treating a disease, is sufficient to affect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “conjugation site” refers to the site where a covalent linkage is formed between two macromolecules, mostly terminal-to-sidechain branched conjugations, and occasionally molecular head-to-tail linear conjugations.

The present disclosure demonstrates that several secretory proteins such as Klotho (KL), cation-independent mannose-6-phosphate receptor (CI-MPR), Klotho, interleukin (IL)-1β, IL-1 receptor antagonist (IL-1RA), TNF-α, etanercept (soluble TNF receptor), galectin-8, epidermal growth factor (EGF), fibroblast growth factor (FGF) 23, glial cell-line derived neurotrophic factor (GDNF), brain-derived neurotrophic factor (BDNF), β-Nerve Growth Factor (β-NGF), neurotrophin-3 (NT3), transforming growth factor-β1 (TGF-β1), activin A, inhibitin A, bone morphogenetic protein (BMP) 4, BMP6, BMP9, BMP10, growth differentiation factor (GDF)-8, GDF-10, GDF-11, and GDF-15 can carry drug-loaded liposomes to the apoptotic cells. Lectins and lectin-associated proteins are able to carry drug-loaded liposomes to the apoptotic cells. KL can act like a lectin and bind glycoproteins, glycolipids and monogangliosides on cell membranes through a glycan motif present such as gangliosides. The glycan-binding function also evidenced the lectin property of CI-MPR. Additionally, aforementioned growth factors (BDNF, NGF, NT3, activin A, BMP6, BMP9, GDF-8, GDF-10, GDF-11, EGF), and cytokine-related factors (IL-1β, TNF-α, galectin-8, and soluble TNF receptor 2) also demonstrated the lectin property.

The present disclosure shows that the combinations of KL and CI-MPR with secretory proteins such as P-selectin can exert synergistic effect on drug delivery to peripheral sites such as the liver. More importantly, the delivery capability of these combinations is not limited thereto but can also cross biological barriers.

Liposomes provided herein include unilamellar liposomes, multilamellar liposomes and multivesicular liposomes. Liposomes provided herein may be composed of positively charged, negatively charged or neutral phospholipids.

A liposome used in the invention can be provided by various known methods. For example, a phospholipid such as the neutral phospholipid dioleoylphosphatidylcholine (DOPC), Dipalmitoyl Phosphatidylcholine (DPPC) and/or EPC, can be dissolved in alcohol or other organic solvent and then mixed with a component for inclusion in the lipid bilayer. The mixture may further include various detergents. Typically, a lipid mixture is vortexed, frozen in a dry ice/acetone bath, and lyophilized overnight. The lyophilized preparation is stored at −20° C. or less for extended periods of time. When required the lyophilized liposomes are reconstituted.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 minutes to 2 hours, depending on the desired volume of the liposomes. The composition can be dried further in a desiccator under vacuum. The dried lipids generally are discarded after about 1 week because of a tendency to deteriorate with time.

Micelle structure will itself be determined, in large part, by the types and compositions of polymer molecules used to form the micelle and the solvent environment of the micelle. In some embodiments, micelles are fabricated using non-ionic triblock co-polymers consisting of both hydrophilic and hydrophobic monomer units. In embodiments of the present disclosure, a poloxamer, a triblock copolymer of poly(ethylene oxide)-poly(propylene oxide)-poly(ethylene oxide) (PEO-PPO-PEO) is used. In some embodiments, the micelles of this disclosure can be prepared using PEG-PLA polymers of a variety of block sizes (e.g., a block size within a range as noted) and in a variety of ratios (e.g., PEG:PLA of about 1:10 to about 10:1, or any integer ratio within said range).

The secretory protein recited herein may be conjugated into the vesicle through a supplement of functional-group labeled lipid using shear force-based methods (Yu B, Lee R J, Lee L J. Microfluidic methods for production of liposomes. Methods Enzymol. 2009; 465:129-141; Jeong D, Jo W, Yoon J, et al. Nanovesicles engineered from ES cells for enhanced cell proliferation. Biomaterials. 2014; 35 (34): 9302-9310).

The protein-vesicle conjugate of the present disclosure can be used to treat or diagnose any disease requiring the administration of a diagnostic agent or a therapeutic agent. Any suitable agent or therapeutic agent can be used with the vesicles of the present invention. In addition, the protein-vesicle conjugate is useful for the treatment of infection by pathogens such as viruses, bacteria, fungi, and parasites. Other diseases can be treated using the vesicles of the present invention.

In some embodiments, the protein-conjugated vesicle can deliver a lipid, a protein, DNA, RNA, a therapeutic agent or a nanomaterial. In some embodiments, the therapeutic agent is a cell survival enhancing agent (or a cell death suppressing agent). The delivery of a cell survival enhancing agent (or a cell death suppressing agent to a subject is able to conduct a drug-mediated rescue of tissue injury.

In one embodiment, the agent is a diagnostic agent or a therapeutic agent. In one embodiment, the agent is an autophagic or apoptotic drug. In some embodiments, examples of the agent include, but are not limited to, an anti-pathogen drug, an autophagy inhibitor, an enzyme inhibitor, a cell signaling inhibitor, a diagnostic contrast agent, a cell survival enhancing agent (or a cell death suppressing agent), a cell survival suppressing agent (or a cell death enhancing agent), a cell (such as stem cell and progenitor cell), a cell component, an organelle, a cytotoxic agent, an antitumor drug, a toxin or an antibody a lipid, a protein, DNA, RNA, a therapeutic agent and a nanomaterial. In one embodiment, the antagonist of the aforementioned first protein or second protein (such as the soluble form, corresponding ligand and the neutralizing and blocking antibody) is able to serve as antidotes to reduce the vesicle-targeting to autophagic and apoptotic cells and autophagic and apoptotic cells-containing tissues. In some embodiments, the agent is bardoxolone methyl, chloroquine, quinine, hydrochloroquine, sorafenib, sunitinib, Hsp90 inhibitor, metformin or crizotinib. In some embodiments, the RNA may be one or more of small interfering RNA (siRNA), small hairpin RNAs (shRNA), microRNA (miRNA), messenger RNA (mRNA), transfer RNA (tRNA), and ribosomal RNA (rRNA), antisense RNA, guide RNA (gRNA), ribozyme and RNA aptamers.

In some embodiments, the agent is a cell survival suppressing agent, cell death enhancing agent, or antitumor agent. The delivery of a cell survival suppressing agent (cell death enhancing agent) or antitumor agent is able to reduce target cell survival of those tissues containing naturally occurred autophagy and apoptotic cells such as tumors or reduce the selected specific tissue wherein the autophagy and apoptotic cells are artificially induced in the specific tissues using cytotoxic agents such as a drug, a toxin or an antibody against tissue-specific proteins.

In some embodiments, the therapeutic agent is a stem cell or a progenitor cell. The delivery of stem cells and progenitor cells is able to exert protective physiological functions and rescue autophagic and apoptotic cell-containing tissues.

The protein-vesicle conjugate can be formulated in a variety of different manners known to one of ordinary skill. Pharmaceutically acceptable carriers are determined in part by the particular composition being administered, as well as by the particular method used to administer the composition. Accordingly, a wide variety of suitable formulations of pharmaceutical compositions of the present invention (see, e.g., Remington's Pharmaceutical Sciences, 20.sup.th ed., 2003, supra) exist. Effective formulations include oral and nasal formulations, formulations for parenteral administration, and compositions formulated for extended release.

In some embodiments, the protein-vesicle conjugate or pharmaceutical composition of the invention can be administered to the patient in a variety of ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

For purposes of administration, for example, parenteral administration, sterile aqueous solutions of water-soluble salts (e.g., NaCl) can be employed. Additional or alternative carriers may include sesame or peanut oil, as well as aqueous propylene glycol. Aqueous solutions may be suitably buffered, if necessary, and the liquid diluent can first be rendered isotonic with sufficient saline or glucose. These aqueous solutions are especially suitable for intravenous, intramuscular, subcutaneous, intraperitoneal, and intratumoral (IT) injection.

Formulations suitable for oral administration can consist of liquid solutions, such as an effective amount of a compound of the present invention suspended in diluents, such as water, saline or PEG 400, capsules, sachets, depots or tablets, each containing a predetermined amount of the active ingredient, as liquids, solids, granules or gelatin, suspensions in an appropriate liquid, suitable emulsions, and patches. The liquid solutions recited can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In one embodiment, before administration of the vesicle, the method additionally comprises a step of administering an autophagic and/or apoptotic inducing agent to a target cell or tissue. By using the step, the target cell or tissue would become autophagic or apoptosis so that the vesicle of the invention can target to the autophagic and/or apoptotic cell or tissue and deliver the interested agent thereto. For example, an anti-obesity antibody can be administered to a subject first to render adipose cells or tissues autophagic and/or apoptotic, then the vesicle with an anti-obesity drug is administered to target the autophagic and/or apoptotic adipose cells or tissues so that the adipose cells or tissues can be further damaged by the anti-obesity drug.

Autophagy is a lysosomal degradation pathway that is essential for survival, differentiation, development, and homeostasis. The delivery of an agent or a therapeutic agent with the vesicle of the invention to autophagic cells is directed to a disease associated with autophagy deregulation. The disease associated with autophagy deregulation includes but is not limited to, trauma, exposure to chemical and physical toxic factors, genetic disease, age-related disease, cardiovascular disease, infectious disease, neoplastic disease, neurodegenerative disease, metabolic disease, aging (when ATG5 is overexpressed in the entire organism), obesity (when ATG7 or the pro-autophagic transcription factor EB [TFEB] are overexpressed in hepatocytes), cancer (when beclin 1 is expressed in KRAS-induced lung adenomas), neurodegeneration induced by β-amyloid or α-synuclein or toxicity (when TFEB or beclin 1 are overexpressed in the brain or when cystatin B, an inhibitor of lysosomal cysteine proteases, is knocked out), myodegenerative conditions (when TFEB or beclin 1 are targeted to the skeletal muscle), and chronic lung inflammation caused by cystic fibrosis (when beclin 1 is expressed in the lung).

Apoptosis is controlled by the integration of multiple pro- and anti-apoptotic signals. The delivery of an agent or a therapeutic agent with the vesicle of the invention to apoptotic cells is directed to a disease associated with apoptosis alteration. Diseases associated with apoptosis alteration include but are not limited to, trauma, exposure to chemical and physical toxic factors, genetic disease, age-related disease, age-related disease, cardiovascular disease, infectious disease, neoplastic disease, neurodegenerative disease, metabolic disease, aging, obesity, cancer, neurodegeneration induced by β-amyloid or a-synuclein (alzheimer, parkinson, huntington, amyotrophic lateral sclerosis) or toxicity, myodegenerative conditions, or chronic lung inflammation caused by cystic fibrosis, cardiovascular disorder (such as ischemia, heart failure and infectious disease) and autoimmune disease (systemic lupus erythematosus, autoimmune lymphoproliferative syndrome, rheumatoid arthritis and thyroiditis).

Pyroptosis is a form of programmed cell death characterized by the activation of inflammatory responses, primarily mediated by the gasdermin family of proteins. Unlike apoptosis, which is a non-inflammatory and controlled cell death process, pyroptosis results in cell lysis and the release of pro-inflammatory cytokines and cellular contents, which can lead to local and systemic inflammation. This mechanism plays a crucial role in the body's defense against infections by eliminating infected cells and activating immune responses. However, dysregulated pyroptosis is implicated in various diseases, including sepsis, atherosclerosis, and neurodegenerative disorders, where excessive inflammation can cause tissue damage and exacerbate disease progression. Therapeutic strategies targeting the key molecules involved in pyroptosis, such as caspase-1 and gasdermin D, offer potential for mitigating the detrimental effects of uncontrolled inflammation, providing a promising avenue for treating inflammatory diseases. As caspse-1 activation can lead to apoptosis, caspase-1 inhibitor or siRNA could theoretically be used to rescue apoptosis-associated diseases.

Although the invention has been recited with reference to preferred embodiments and examples thereof, the scope of the present invention is not limited only to those recited embodiments. As will be apparent to persons skilled in the art, modifications and adaptations to the recited invention can be made without departing from the spirit and scope of the invention, which is defined and circumscribed by the appended claims. The following examples are provided for the intent of illustrating embodiments and advantages of the invention and are not intended to limit the scope thereof.

### EXAMPLES

### Example 1: Relative Fluorescence Levels of Mouse Liver. Injection of Liposomes Loaded with Fluorescein and Conjugated with Secretory Proteins can Effectively Target To the Thioacetamide (TAA)-Injured Mouse Liver

The preparation of liposomes. The liposomes were prepared by liposome kits (Sigma-Aldrich Co.) and respective lipids. Surface proteins were conjugated to the liposomes by incorporating functional-group labeled lipids and utilizing shear force-based methods (Yu, B.; Lee, R. J.; Lee, L. J. Microfluidic methods for production of liposomes. Methods Enzymol 2009, 465, 129-141, doi: 10.1016/S0076-6879 (09) 65007-2; Jeong, D.; Jo, W.; Yoon, J.; Kim, J.; Gianchandani, S.; Gho, Y. S.; Park, J. Nanovesicles engineered from ES cells for enhanced cell proliferation. Biomaterials 2014, 35, 9302-9310, doi: 10.1016/j.biomaterials.2014.07.047). The protein conjugation procedure followed the manufacturer's provided methods.

Synthesis and conjugation of liposomes. The liposomes were synthesized using established methods as recited previously (Chen, Y. L.; Chen, Y. S.; Chan, H.; Tseng, Y. H.; Yang, S. R.; Tsai, H. Y.; Liu, H. Y.; Sun, D. S.; Chang, H. H. The use of nanoscale visible light-responsive photocatalyst TiO2-Pt for the elimination of soil-borne pathogens. PloS one 2012, 7, e31212, doi: 10.1371/journal.pone.0031212; Wu, C. H.; Kuo, Y. H.; Hong, R. L.; Wu, H. C. alpha-Enolase-binding peptide enhances drug delivery efficiency and therapeutic efficacy against colorectal cancer. Sci Transl Med 2015, 7, 290ra291, doi: 10.1126/scitranslmed.aaa9391).

The recombinant proteins. The recombinant proteins used in this study, such as recombinant MPRs (cation-independent mannose-6-phosphate receptor: CI-MPR; cation-dependent mannose-6-phosphate receptor: CD-MPR), were obtained from R and D Systems Co.

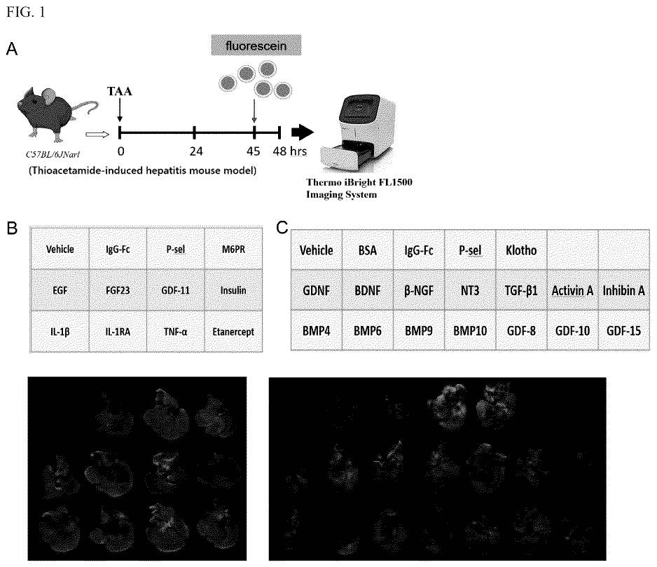

Based on the previously established mouse model of TAA-induced hepatitis (U.S. Pat. No. 10,584,154 B2), our current study demonstrates that liposomes loaded with a fluorescent dye and conjugated with secretory proteins, such as CI-MPR, P-selectin, Klotho, interleukin (IL)-1β, IL-1 receptor antagonist (IL-1RA), TNF-α, etanercept (soluble TNF receptor), epidermal growth factor (EGF), fibroblast growth factor (FGF) 23, glial cell-line derived neurotrophic factor (GDNF), brain-derived neurotrophic factor (BDNF), β-Nerve Growth Factor (β-NGF), neurotrophin-3 (NT3), transforming growth factor-β1 (TGF-β1), activin A, inhibitin A, bone morphogenetic protein (BMP) 4, BMP6, BMP9, BMP10, growth differentiation factor (GDF)-8, GDF-10, GDF-11, and GDF-15, can deliver higher levels of fluorescent dye into the TAA-damaged mouse liver, as compare to the conjugation of control proteins BSA and IgG-Fc (FIG. 1). Additionally, another control growth factor, insulin, did not show the same delivery capability as the aforementioned factors. The targeting efficiency was visualized using a fluorescent imaging system (Thermo iBright FL1500) with liposomes labeled with a commercially available dye (fluorescein; Sigma-Aldrich Co.). The captured images confirmed the successful delivery of the fluorescent dye into the inflamed mouse brain following TAA treatments [FIG. 1A, experimental settings; 1B and 1C, The protein used for liposome conjugation (upper panels), and fluorescent images of mouse liver (lower panels)].

### Example 2: Quantitative Analysis of Relative Fluorescence Levels of Mouse Liver. Injection of Liposomes Loaded with Fluorescein and Conjugated with Secretory Proteins Can Effectively Target the Thioacetamide (TAA)-Injured Mouse Liver

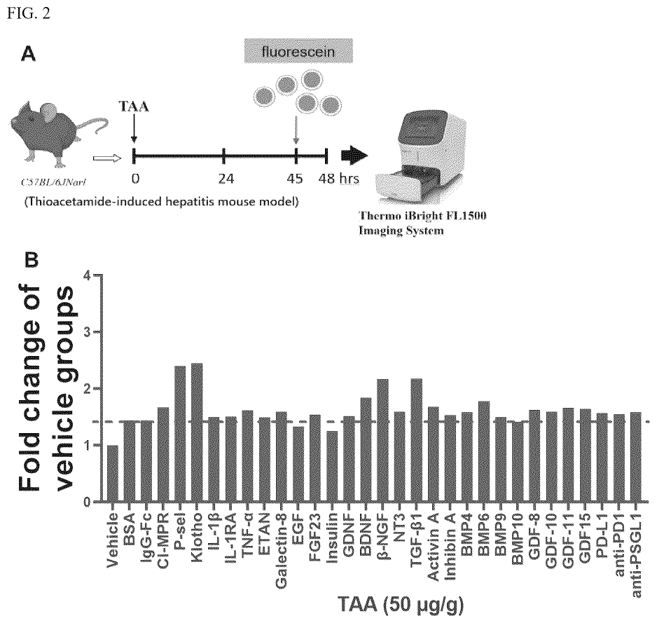

Specialized liposomes, engineered with protein conjugates, were tested to selectively target inflamed and injured cells in the mouse liver. The targeting efficiency was visualized using a fluorescent imaging system (Thermo iBright FL1500) with liposomes labeled using a commercially available dye (fluorescein; Sigma-Aldrich Co.). The quantified fluorescent intensity of captured images demonstrated successful delivery of the fluorescent dye into the inflamed mouse brain after restraint stress (FIGS. 2A, experiment settings: 2B, quantified fluorescent intensity of fluorescein-delivered mouse liver)

### Example 3: Caspase-1 siRNA-Loaded Engineered Liposomes with Lectin Conjugations Effectively Alleviate TAA-Induced Liver Damage in Mice

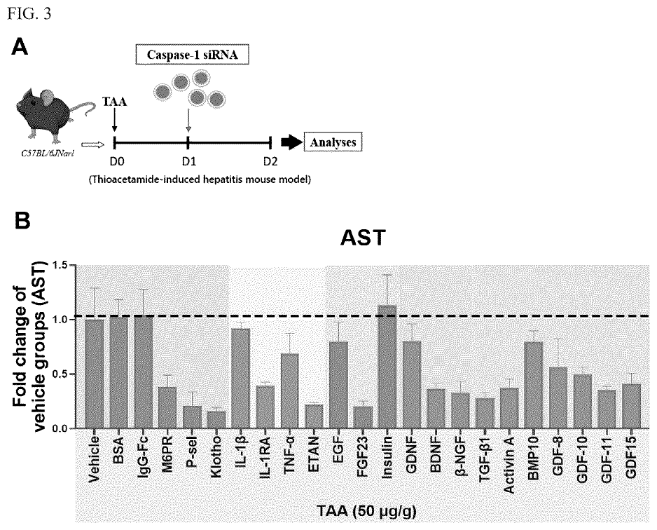

Engineered liposomes with lectin conjugations were designed to selectively target inflamed and injured cells in vivo. We employed a TAA-induced hepatitis mouse model to investigate the potential of caspase-1 siRNA-loaded engineered liposomes with lectin conjugations to alleviate TAA-induced liver damage. TAA toxicity induces elevated circulating liver damage markers, such as aspartate aminotransferase (AST) and alanine transaminase (ALT), and results in thrombocytopenia in mice (Sci Rep 2019 Nov. 25; 9:17497. doi: 10.1038/s41598-019-53977-7). We analyzed AST levels in TAA-treated mice using a clinical biochemistry analysis system (COBAS INTEGRA 800, Roche Taiwan, Taipei, Taiwan), following previously recited methods (Sci Rep 2019 Nov. 25; 9:17497. doi: 10.1038/s41598-019-53977-7). In line with the fluorescent dye delivery results, caspase-1 siRNA-loaded engineered liposomes conjugated with secretory proteins effectively targeted the mouse liver and alleviated TAA-induced liver damage (FIG. 3A, experimental settings; FIG. 3B, circulating AST levels). The AST levels were lower than those in the control groups, where the siRNA was delivered using non-functional proteins such as BSA and IgG-Fc, indicating a significant protective effect against TAA-induced liver damage (FIG. 3 dashed lines, AST level of TAA+vehicle treated groups was normalized to 1-fold). These findings suggest that engineered liposomes conjugated with the aforementioned secretory proteins and proteins (except insulin; FIG. 3B) are an ideal platform for delivering drugs, such as siRNA, to stressed cells and ameliorating TAA-induced damage in vivo.

### Example 4: Liposomes Loaded with Caspase-3 Inhibitor Z-DEVD-FMK and Conjugated with Multiple Secretory Proteins (e.g., CI-MPR+KL, CI-MPR+P-Selectin, and KL+P-Selectin) Significantly Alleviated TAA-Induced Liver Damage in Mice Compared to Cases where Each Lectin was Used Individually

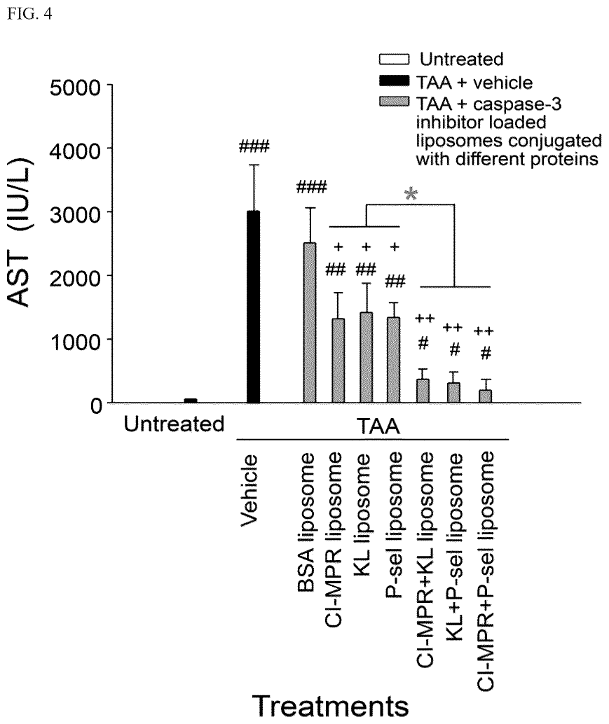

Based on the previously established mouse model of TAA-induced hepatitis (U.S. Pat. No. 10,584,154 B2), our current study reveals that liposomes loaded with an apoptosis inhibitor (specifically, a caspase-3 inhibitor Z-DEVD-FMK) and conjugated with secretory proteins (such as CI-MPR, KL, P-selectin, CI-MPR+KL, CI-MPR+P-selectin, and KL+P-selectin) can effectively mitigate the elevated levels of AST and hepatitis in TAA-treated mice (FIG. 4). It is worth noting that the therapeutic impact of liposomes carrying the drug, when conjugated with multiple secretory proteins (e.g., CI-MPR+KL, CI-MPR+P-selectin, and KL+P-selectin), is significantly more pronounced compared to each individual lectin treatment. This suggests a synergistic effect when employing a combination of these secretory proteins in conjunction with the liposomes.

1. A protein-vesicle conjugate, comprising:
(a) one or more secretory proteins or a fragment thereof expressed on or conjugated to a surface of a vesicle, and/or one or more antibodies specific to the one or more secretory proteins of the fragment thereof and expressed on or conjugated to the surface of the vesicle; and
(b) optionally an agent,
wherein the one or more secretory proteins or the fragment thereof is selected from cation-independent mannose-6-phosphate receptor (CI-MPR), Klotho,
programmed death-ligand 1 (PD-L1), intercellular adhesion molecule 1 (ICAM-1; CD54), Toll like receptor 3 (TLR3), IL-1β, IL-1 receptor antagonist (IL-1RA), TNF-α, etanercept (soluble TNF receptor), galectin-8, EGF, FGF23, GDNF, BDNF, β-NGF, NT3, TGF-β1, activin A, inhibitin A, BMP4, BMP6, BMP9, BMP10, GDF-8, GDF-10, GDF-11, GDF-15, PSGL-1, PD1 and a fragment thereof.

2. The protein-vesicle conjugate of claim 1, wherein the one or more antibodies specific to the one or more secretory proteins or the fragment thereof is selected from anti-PSGL-1 and anti-PD1 antibodies.

3. The protein-vesicle conjugate of claim 1, wherein the agent is separated from the secretory proteins or the fragment thereof and encapsulated within the vesicle or attach to an outer surface of the vesicle.

4. The protein-vesicle conjugate of claim 1, wherein the vesicle is a liposome or a micelle.

5. The protein-vesicle conjugate of claim 1, which is artificially engineered.

6. The protein-vesicle conjugate of claim 1, which is cell-derived.

7. The protein-vesicle conjugate of claim 1, further comprising one or more additional lectins or fragment thereof expressed on or conjugated to the surface of the vesicle, wherein the one or more additional lectins or the fragment thereof is selected from P-selectin, E-selectin, L-selectin, P-selectin-ligand-1 (PSGL-1), cation-dependent mannose-6-phosphate receptor (CD-MPR), macrophage mannose receptor (MMR; CD206), galectin 1, galectin 3, annexin V, CD31, integrin αLβ2, VE-cadherin, CD44, CD300a, CD47, CD36, Siglec (CD22), thrombospondin 1 (TSP1), CLEC2, Toll like receptor 4 (TLR4) and a fragment thereof.

8. The protein-vesicle conjugate of claim 8, which comprises CI-MPR in combination with Klotho, CI-MPR in combination with P-selectin, or Klotho in combination with P-selectin.

9. The protein-vesicle conjugate of claim 1, wherein the agent is a diagnostic contrast agent, a cell survival enhancing agent, a cell survival suppressing agent, a cell component, an organelle, a cell, a cytotoxic agent, an antitumor drug, a toxin or an antibody a lipid, a protein, DNA, RNA, a therapeutic agent or a nanomaterial.

10. A pharmaceutical composition comprising the protein-vesicle conjugate of claim 1 and a pharmaceutically acceptable carrier.

11. A pharmaceutical composition comprising the protein-vesicle conjugate of claim 1, and antagonists to the one or more secretory proteins or a fragment thereof.

12. The composition of claim 12, wherein the antagonists to the one or more secretory proteins or a fragment thereof are soluble forms of the secretory proteins or fragments thereof, corresponding ligands to the secretory proteins or fragments thereof, or antibodies to the secretory proteins or fragments thereof.

13. The composition of claim 11, wherein the antagonists to the one or more secretory proteins or a fragment thereof reduce the protein-vesicle conjugate targeting to autophagic and/or apoptotic cells and autophagic and/or apoptotic cells-containing tissues.

14. A method for delivery of an agent to an autophagic and/or apoptotic cell and a tissue containing the cell, comprising administering the protein-vesicle conjugate of claim 1 to a subject.

15. The method of claim 15, wherein before administration of the protein-vesicle conjugate, the method further comprises a step of administering an autophagic and/or apoptotic-inducing agent to a target cell or a target tissue.

16. The method of claim 15, wherein the delivery of the agent with the protein-vesicle conjugate of claim 1 to the autophagic cell is directed to a disease associated with autophagy deregulation.

17. The method of claim 17, wherein the disease associated with autophagy deregulation is trauma, exposure to chemical and physical toxic factors, genetic disease, age-related disease, cardiovascular disease, infectious disease, neoplastic disease, neurodegenerative disease, metabolic disease, aging, obesity, cancer, neurodegeneration induced by β-amyloid or α-synuclein or toxicity, myodegenerative conditions, or chronic lung inflammation caused by cystic fibrosis.

18. The method of claim 15, wherein the delivery of the agent with the protein-vesicle conjugate of claim 1 to the apoptotic cell is directed to a disease associated with apoptosis alteration.

19. The method of claim 19, wherein the disease associated with apoptosis alteration is trauma, exposure to chemical and physical toxic factors, genetic disease, age-related disease, age-related disease, cardiovascular disease, infectious disease, neoplastic disease, neurodegenerative disease, metabolic disease, aging, obesity, cancer, neurodegeneration induced by β-amyloid or α-synuclein or toxicity, myodegenerative conditions, or chronic lung inflammation caused by cystic fibrosis, or autoimmune disease.

20. The method of claim 20, wherein the neurodegenerative disorder is Alzheimer's disease, Parkinson's disease, Huntington's disease, Amyotrophic Lateral Sclerosis or stroke, the cardiovascular disorder is ischemia, heart failure or infectious disease and the autoimmune disease is systemic lupus erythematosus, autoimmune lymphoproliferative syndrome, rheumatoid arthritis or thyroiditis.
